(19) United States
(12) Patent Application Publication
(10) Pub. No.: US 2012/0196892 A1
(43) Pub. Date: Aug. 2, 2012

(54) NOVEL CASPASE-2 INHIBITORS

(75) Inventor: David CHAUVIER, Parma (IT)

(73) Assignee: CHIESI FARMACEUTICI S.p.A., Parma (IT)

(21) Appl. No.: 13/362,104

(22) Filed: Jan. 31, 2012

(30) Foreign Application Priority Data
Feb. 1, 2011 (EP) 11152892.3

(51) Int. Cl. A61K 31/47; A61P 25/00; C07D 215/48

(52) U.S. Cl. 514/311; 546/169

(57) ABSTRACT

Novel compounds of formula (I) and slats thereof and pharmaceutical compositions which contain such a compound or salt are useful for inhibiting pro-apoptotic caspase-2 and for preventing and/or treating diseases and injuries where caspase-2 activity is implicated, in particular neonatal brain ischemia.

### CROSS REFERENCES TO RELATED APPLICATIONS

This application claims priority to European Patent Application No. 11152892.3 filed on Feb. 1, 2011, which is incorporated herein by reference in its entirety.

### BACKGROUND OF THE INVENTION

1. Field of the Invention

The present invention is in the field of medicinal biology and chemistry and relates to novel compounds and pharmaceutical compositions, which inhibit pro-apoptotic caspase-2. The present invention also relates to methods for preventing and/or treating diseases and injuries where caspase-2 activity is implicated by administering such a compound or pharmaceutical composition.

2. Discussion of the Background

Neuronal cell death occurs during embryogenesis to remove excess of neurons to ensure appropriate pre- and post-synaptic connections and to allow formation of a functional adult brain. Besides post-mitotic death related to normal ageing, environmental or genetic mutational factors may induce neuronal death in the adult human during acute injuries (for instance, hypoxia-ischemia, stroke, spinal cord injury, trauma) or chronic neurodegenerative diseases. Cell death associated with these disorders may occur by three distinct mechanisms, exhibiting morphological and biochemical features of necrosis, autophagy or apoptosis. Both physiological and pathological neuronal deaths are often associated with defective apoptosis regulation, and signaling pathways that lead to this active cell suicide mechanism may be divided into cysteinyl aspartate-specific protease (caspase)-dependent versus caspase-independent pathways in mammalian cells.

Neuronal apoptosis is an active cell suicide mechanism that can be divided into sequential phases, including initiation, decision, execution and degradation. This cascade of events is driven by the activation of specific machinery that involves both the activation of cysteine-dependent aspartate-specific proteases (caspases) and the mitochondrion which may act as a decisive (or amplifier) regulatory organelle. Indeed, mitochondrial alterations include loss of mitochondrial inner membrane electrochemical gradient and release of apoptogenic factors such as cytochrome c Smac/Diablo and Apoptosis Inducing Factor. Once released from mitochondria, these effectors trigger caspase-dependent and/or caspase-independent cytoplasmic and nuclear dismantling. Hence, mitochondrial factors combined with caspases contribute to the degradation phase of apoptosis, resulting in cell shrinkage, nuclear condensation, emission of apoptotic bodies, and appearance of “eat-me” signals such as phosphatidyl-serines translocation to the outer leaflet of the plasma membrane before phagocytosis.

So far, several caspases have been identified in humans. In particular, there are two types of apoptotic caspases: initiator (apical) caspases and effector (executioner) caspases. Initiator caspases (e.g., caspase-2, caspase-8, caspase-9, caspase-10) cleave inactive pro-forms of effector caspases, thereby activating them. Effector caspases (e.g., caspase-2, caspase-6, caspase-7) in turn cleave other protein substrates within the cell, to trigger the apoptotic process.

Several caspase inhibitors have been disclosed in the art. Compounds able to inhibit caspase-2 activity have been reported in WO 2004/103389, WO 2005/105829 and WO 2006/056487, all of which are incorporated herein by reference in their entireties.

In particular, the pentapeptide 5-(2,6-difluoro-phenoxy)-3(R,S)-{2(S)-[2(S)-(3-methoxycarbonyl-2(S)-{3-methyl-2(S)-[(quinoline-2-carbonyl)-amino]butyrylamino}-propionylamino)-3-methyl-butyrylamino]-propionylamino}-4-oxo-pentanoic acid methyl ester is a selective caspase-2 inhibitor, disclosed in WO 2005/105829, currently under development for the treatment of neonatal brain injury. This compound has the formula shown below:

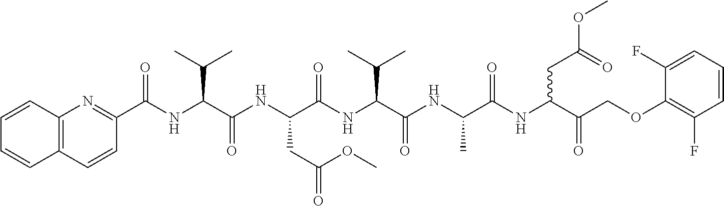

It has also been referred to in the art as quinoline-2-carbonyl-(S)-Val-(S)-Asp(OMe)-(S)-Val-(S)-Ala-(R,S)-Asp(OMe)-CH₂OC₆H₃(2,6-F₂) or TRP 601.

However, there still remains a need for caspase-2 inhibitors with improved properties, in particular in terms of pharmacokinetics and ADME (adsorpion, distribution, metabolism and excretion) properties.

In particular, it would be highly advantageous to provide more efficacious caspase-2 inhibitors for use for the treatment of neonatal brain ischemia which is a major cause of neurodevelopmental damage in pre-terms infants and remains a major unmet medical need.

### SUMMARY OF THE INVENTION

Accordingly, it is one object of the present invention to provide novel capase-2 inhibitors.

It is another object of the present invention to provide novel caspase-2 inhibitors with improved properties, in particular in terms of pharmacokinetics and ADME (adsorpion, distribution, metabolism and excretion) properties.

It is another object of the present invention to provide novel pharmaceutical compositions which contain such a capase-2 inhibitor.

It is another object of the present invention to provide novel methods of preventing and/or treating a disease or injurie where caspase-2 activity is implicated by administering such a compound or pharmaceutical composition.

It is another object of the present invention to provide novel methods of preventing and/or treating neonatal brain ischemia by administering such a compound or pharmaceutical composition.

These and other objects, which will become apparent during the following detailed description, have been achieved by the inventors' discovery that compounds of general formula (I) described above are effective as capase-2 inhibitors.

Thus, in a first aspect, the present invention provides caspase-2 inhibitors of general formula (I):

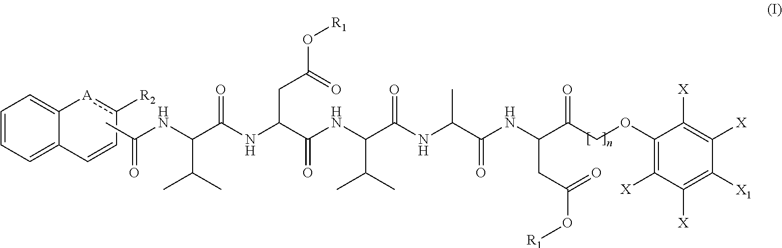

wherein:

n is 0 or 1;

A represents N, N(H), O, S, or N—O (i.e. N-oxide);

[symbol] adjacent to the group A can be a single bond when A is O or S, or a single or double bond when A is N(H) or N, respectively;

each X may be the same or different from each other and is independently selected from halogen atoms, preferably fluorine;

X₁ represents H or a halogen atom;

each R₁ may be the same or different from each other and is independently selected from H and a linear or branched (C₁-C₄) alkyl group, preferably from H, methyl and ethyl; and

R₂ is absent, when [symbol] is a double bond, and is H, when [symbol] is a single bond.

In a second aspect, the present invention provides pharmaceutical compositions comprising a compound of general formula (I) as active ingredient, and, optionally, one or more pharmaceutically acceptable excipients.

In a third aspect, the present invention provides a compound of general formula (I) for use as a medicament.

In a fourth aspect the present invention provides a compound of general formula (I) for use for the prevention or treatment of a disease where caspase-2 activity is implicated, such as neonatal brain injury.

In a fifth aspect the present invention provides the use of a compound of general formula (I) in the preparation of a medicament for the prevention or treatment of a disease where caspase-2 activity is implicated, such as neonatal brain injury.

In a sixth aspect the present invention provides a method for the prophylaxis or treatment of a disease where caspase-2 activity is implicated, such as neonatal brain injury, said method comprising the administration of a therapeutically effective amount of a compound of general formula (I).

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a graph showing the in vitro activity of TRP 701 versus other compounds.

### DETAILED DESCRIPTION OF THE PREFERRED EMBODIMENTS

Unless defined otherwise, all technical and scientific terms used herein have the same meaning as it is commonly understood by one of skill in the art to which this subject matter belongs.

The term “halogen atoms” includes fluorine, chlorine, bromine and iodine.

The expression “linear or branched (C₁-C₄) alkyl” refers to straight-chained and branched alkyl groups wherein the number of constituent carbon atoms is in the range 1 to 4. Particular examples of alkyl groups are methyl, ethyl, n-propyl, isopropyl, and t-butyl.

For “dosage”, it is meant the unitary amount of drug to be administered. “An effective amount of a compound for treating a particular disease” is an amount that is sufficient to ameliorate, or in some manner reduce, the symptoms associated with the disease.

The term “high level of chemical purity” refers to a compound wherein the total amount of readily detectable impurities as determined by standard methods of analysis, such as thin layer chromatography (TLC) or high performance liquid chromatography (HPLC), is less than 5%, advantageously less than 2.5%, preferably less than 1.0, more preferably less than 0.5% w/w.

The term “prevention” means an approach for reducing the risk of onset of a disease.

The term “treatment” means an approach for obtaining beneficial or desired results, including clinical results. Beneficial or desired clinical results can include, but are not limited to, alleviation or amelioration of one or more symptoms or conditions, decrease in the extent of disease, stabilized (i.e. not worsening) state of disease, preventing spread of disease, delay or slowing of disease progression, amelioration or palliation of the disease state, and remission (whether partial or total), whether detectable or undetectable. The term can also mean prolonging survival as compared to expected survival if not receiving treatment.

Thus, the present invention is directed to caspase-2 inhibitors of general formula (I):

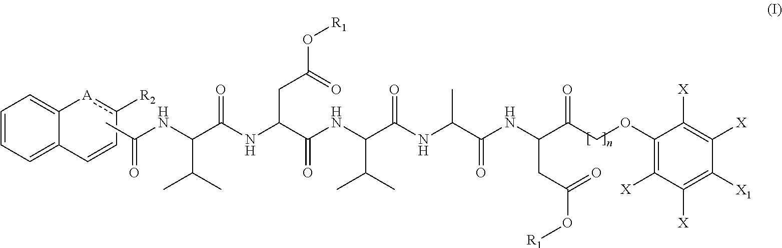

wherein:

n is 0 or 1;

A represents N, N(H), O, S, or N—O (i.e. N-oxide);

[symbol] adjacent to the group A can be a single bond when A is O or S or a single or double bond when A is N(H) or N, respectively;

each X may be the same or different from each other and is independently selected from halogen atoms, preferably fluorine;

X₁ represents H or a halogen atom;

each R₁ may be the same or different from each other and is independently selected from H and a linear or branched (C₁-C₄) alkyl group, preferably from H, methyl and ethyl; and

R₂ is absent, when [symbol] is a double bond, and is H, when [symbol] is a single bond.

When A is N and [symbol] is a double bond, the present invention also encompasses the corresponding N-oxides on the quinoline ring.

Thus, [symbol] adjacent to the group A is a single bond, when A is O, S, or N(H), or a double bond, when A is N or N—O.

In a preferred embodiment of the present invention, A is N and [symbol] is a double bond thereby forming a quinoline ring.

More preferably, the caspase-2 inhibitor is a compound of general formula (II) in which n=1

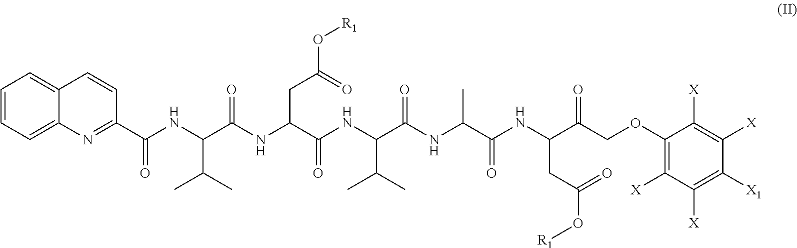

wherein each of X, X₁ and R₁ have the meanings defined above.

Moreover, the invention encompasses pharmaceutically acceptable salts and/or solvates thereof. Pharmaceutically acceptable salts include those in which acidic functions, when present, are reacted with an appropriate base to form, e.g. sodium, potassium, calcium, magnesium, ammonium and choline salts.

When A is N and [symbol] is a double bond, pharmaceutically acceptable salts also include those obtained by reacting the nitrogen of the quinoline ring, functioning as a base, with a strong inorganic or organic acid to form, for example, salts of hydrochloric acid and trifluoromethanesulfonic acid.

The compounds of general formula (I) are derivatives of the amino acid sequence Valinyl-Aspartyl-Vanilyl-Alanyl-Aspartyl and contain asymmetric centers having either the absolute R configuration, corresponding to an L-amino acid residue, or the S configuration corresponding to a D-amino acid residue. The invention includes the optical stereoisomers and mixtures thereof.

Preferably, the asymmetric centers of the valinyl and alanyl amino acid residues have the absolute configuration (S) while that of the aspartyl residue could be (S) or (R,S).

A preferred group of compounds of general formula (II) is that wherein R₁ is H, each of X is F, and X₁ is H.

Accordingly, in one of the preferred embodiments of the present invention, the caspase-2 inhibitor is the compound 2-quinolinylcarbonyl-(S)-Valinyl-(S)-Aspartyl-(S)-Valinyl-(S)-Alanyl-(R,S)-Aspartyl 2,3,5,6-tetrafluorophenyl ester.

Another preferred group of compounds of general formula (II) is that wherein R_{I} is methyl, each of X is F, and X₁ is H.

Accordingly, in one of the preferred embodiments of the present invention, the caspase-2 inhibitor is the compound 5-(2,3,5,6-tetrafluoro-phenoxy)-3(R,S)-{2(S)-[2(S)-(3-methoxycarbonyl-2(S)-{3-methyl-2(S)-[(quinoline-2-carbonyl)-amino]-butyrylamino}-propionylamino)-3-methyl-butyrylamino]-propionylamino}-4-oxo-pentanoic acid methyl ester, also referred to hereinafter with the internal code TRP 701.

Said latter compound can also be called as 2-quinolinylcarbonyl-(S)-Valinyl-(S)-Aspartyl (methyl ester)-(S)-Valinyl-(S)-Alanyl-(R,S)-Aspartyl (methyl ester) 2,3,5,6-tetrafluorophenyl ester or quinoline-2-carbonyl-(S)-Val-(S)-Asp(OMe)-(S)-Val-(S)-Ala-(R,S)-Asp(OMe)-CH₂OC₆H(2,3,5,6-F₄).

Another preferred group of compounds of general formula (II) is that wherein R₁ is ethyl, each of X is F, and X₁ is H.

Accordingly, in one of the preferred embodiments of the present invention, the caspase-2 inhibitor is the compound 2-quinolinylcarbonyl-(S)-Valinyl-(S)-Aspartyl (ethyl ester)-(S)-Valinyl-(S)-Alanyl-(R,S)-Aspartyl (ethyl ester) 2,3,5,6-tetrafluorophenyl ester.

Unless otherwise provided, the present invention includes all of those compounds wherein the ring bearing group A is substituted, in any suitable free position, with the residual portion of the molecule through the amido linkage as represented in formula (I). In other words, the structure:

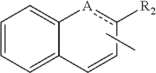

means that the remainder of the molecule may be bonded to:

any of the carbons atoms in the bicyclic ring by replacing the hydrogen atom on that carbon atom with the bond to the remainder of the molecule; or

A, when A is N(H), by replacing the hydrogen atom on N(H) with the bond to the remainder of the molecule.

The compounds of the present invention, being very lipophilic, may readily cross the blood-brain barrier and diffuse into the brain. Moreover, the addition of more halogen atoms, in particular fluorine, on the terminal phenoxy group, make the resulting compounds endowed with a more rapid onset of action in comparison to compounds with less halogen atoms such as TRP 601. Without being limited by the theory, it was afterwards hypothesized that the supplementary halogen atoms on the aromatic ring may even enhance the electron delocalization of the relevant compound which, in turn, makes it more prone to rapid interaction with the active thiol (SH) group of the active site in the pocket of caspase-2.

The compounds of general formula (I) may be prepared by known methods. Some of the processes which can be used are reported in Schemes 1, 2 and 3, wherein A, X, X₁ and R₁ have the above reported meanings.

The conditions and the reagents to be utilized are disclosed in WO 2005/105829, which is incorporated herein by reference in its entirety. However said synthetic pathways should not be viewed as limiting the scope of the synthetic methods available for the preparation of the compounds of the present invention.

The schemes shown below refer to the preparation of the compounds of general formula (I) wherein n=1 and [symbol] is a double bond, but they can be applied as well for the preparation of the compounds wherein n=0 and [symbol] is a single bond, using the suitable intermediates accordingly.

The starting materials and the reagents are known or, if not commercially available per se, can be readily prepared according to known methods.

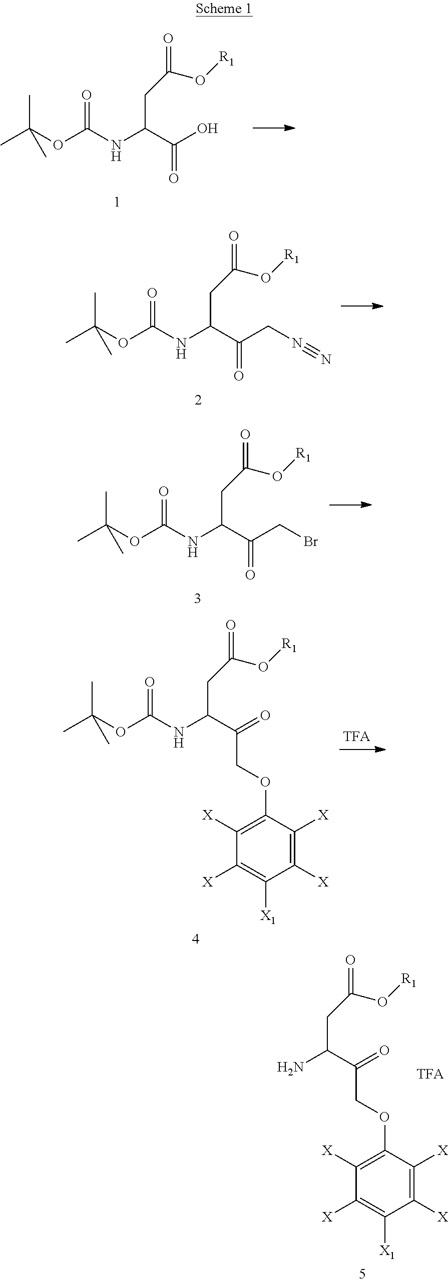

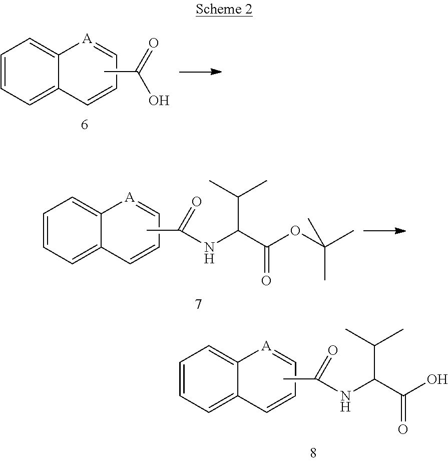

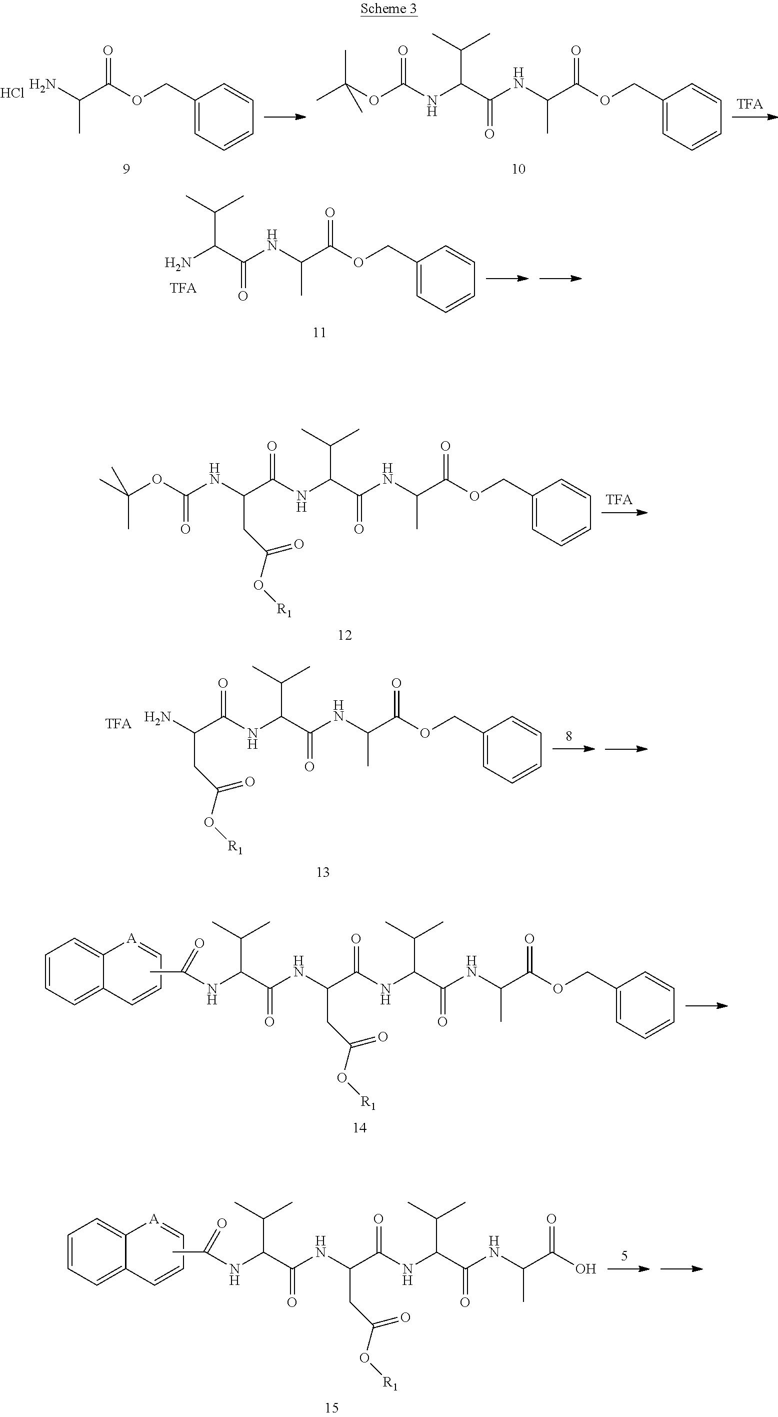

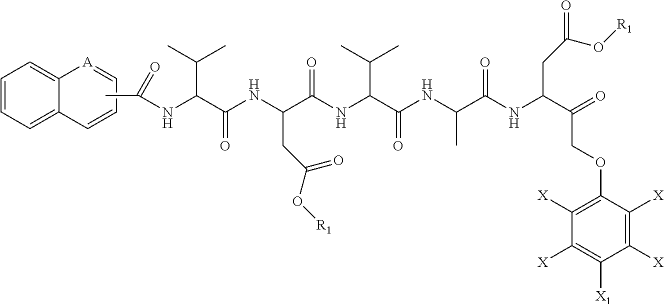

Advantageously, the compounds of general formula (I) are utilized with a high level of chemical purity for the preparation of pharmaceutical compositions, for administration in any convenient way.

Suitable dosages of the compounds of the present invention may easily be established by the attending physician and will depend on the type of patient, the age of the patient, nature of the disease and on the mode of drug delivery. Daily dosages on the order of about 0.01 mg to about 5 mg per kilogram of body weight (mg/kg) may be useful, preferably of about 0.1 to about 3 mg/kg, more preferably of about 0.5 to about 2 mg/kg. These dosages may be achieved by a single daily dosage or by multiple daily dosages of smaller amounts. How long the dosing is continued will also depend on the type of patient, the age of the patient, and the nature of the disease and may be easily determined by the attending physician. For example, when treating the diseases perinatal arterial stroke (PAS), perinatal hypoxic-ischaemic encephalopathy (HIE), and periventricular leucomalacia, it may be preferred to continue the dosing from the time of detection of the disease until some later time as indicated by amelioration, palliation, or remission of the disease state, or even for some time afterwards.

Pharmaceutical compositions may be prepared by admixing a compound of general formula (I) in a suitable dosage and one or more pharmaceutically acceptable excipients. Depending on the nature of the medical disease or condition to be treated and the type of patient, the pharmaceutical compositions may be formulated to be delivered by any suitable route, including oral, intravenous, parenteral, inhalation, intranasal, topical, subcutaneous, intramuscular, rectal, intraperitoneal, intracerebroventricular, intrahippocampal or other intracerebral delivery, intracerebral implantation of instrumentation for mechanical delivery such as of Gelfoam® impregnated with a compound of the invention. Suitable dosage forms include all those known to the skilled person, such as tablets, capsules, powders, sustained release formulations, ointments, gels, creams, suppositories, eye drops, transdermal patches, syrups, solutions, suspensions, aerosols, solutions for nebulizers, nasal sprays etc.

Suitable known excipients, include carriers, diluents, wetting agents, emulsifying agents, binders, coatings, fillers, glidants, lubricants, disintegrants, preservatives, surfactants, pH buffering substances and the like. Examples of excipients are provided in the Handbook of Pharmaceutical Excipients, 5^{th} ed. (2006), Ed. Rowe et al., Pharmaceutical Press, which is incorporated herein by reference in its entirety.

In preferred embodiments the compositions are formulated for delivery by intravenous, subcutaneous, intraperitoneal, or intracerebral routes.

In some embodiments of the present invention, the compositions may be formulated in the form of liposomal solutions or microsuspensions. In other embodiments, they may be formulated in form of aqueous solutions, which may be optionally pH-buffered.

The solvent wherein the compound of general formula (I) should be dissolved may consist of only water or of a mixture of water and a co-solvent, miscible with water, selected from the group consisting of ethanol, propylene glycol, polyethylene glycol, polypropylene glycol, and glycerol or mixtures thereof.

In a preferred embodiment, the composition is formulated in the form of an aqueous solution, optionally pH-buffered comprising a compound of general formula (II) wherein R₁ is H, each of X is F, and X₁ is H, or a salt thereof at a dosage comprised between 0.02 and 0.25 mg/kg, more preferably between 0.05 and 0.2 mg/kg.

The compositions may also comprise, if required, one or more other therapeutic agents, preferably those currently used in the treatment of neonatal diseases.

The compounds of general formula (I) may be used for prophylactic purposes or for the treatment of a wide range of diseases involving caspase-2 activity.

For example, said compounds are advantageously useful for preventing, reducing and treating pathologies characterized by cell death, particularly in hypoxic-ischemic (H-I) brain damages and stroke-like situations brain injuries: for example, global or focal, transient or permanent, adult or neonatal H-I (ischemia with or without hypoxia/hypoglycaemia) with origin at cerebral or heart level, with or without reperfusion, or MCAO (Middle Cerebral Artery Occlusion).

Preferably, the compounds of the present invention are utilized for the treatment of neonatal brain injury that encompasses Perinatal Arterial Stroke (PAS), Perinatal Hypoxic-Ischaemic Encephalopathy (HIE), and Periventricular Leucomalacia (white matter injury) in premature babies, more preferably for the treatment of Perinatal Hypoxic-Ischaemic Encephalopathy.

The compounds of the invention may also be useful for:

preventing and/or treating apoptosis during chronic degenerative diseases e.g. neurodegenerative disease including Alzheimer's disease, Huntington's disease, Parkinson's disease, Multiple sclerosis, amyotrophic lateral sclerosis, spinobulbar atrophy, prion disease, and dementia; or

preventing and/or treating retinal pericyte apoptosis, retinal neurons apoptosis glaucoma, retinal damages resulting from local ischemia, and diabetic retinopathy; or

preventing and/or treating epilepsy; or

preventing and/or treating apoptosis during spinal cord injury, or to prevent and/or treat apoptosis resulting from traumatic brain injury, retinal ischemia; or

preventing and/or treating apoptosis during pathological situations of focal cerebral ischemia; or

providing cerebroprotective effect; or

preventing and/or treating cytotoxic T cell and natural killer cell-mediated apoptosis associated with autoimmune disease and transplant rejection; or

preventing and/or treating cell death of cardiac cells including heart failure, cardiomyopathy, viral infection or bacterial infection of heart, myocardial ischemia, myocardial infarct, and myocardial ischemia, and coronary artery by-pass graft; or

preventing and/or treating mitochondrial drug toxicity e.g. as a result of chemotherapy or HIV therapy; or

preventing and/or treating cell death during viral infection or bacterial infection; or

preventing and/or treating inflammation or inflammatory diseases, inflammatory bowel disease; sepsis, and septic shock; or

preventing cell death from follicule to ovocyte stages, from ovocyte to mature egg stages and sperm (for example, methods of freezing and transplanting ovarian tissue, artificial fecundation); or

preserving fertility in women and men after chemotherapy; or

preserving fertility in females and males animals, or to prevent and/or treat, macular degenerescence and glaucoma; or

preventing and/or treat acute hepatitis, chronic active hepatitis, hepatitis-B, and hepatitis-C; or

preventing and/or treating hair loss, and said hair loss due-to male-pattern baldness, radiation, chemotherapy, or emotional stress; or

treating or ameliorating skin damage (due to exposure to high level of radiation, heat, burns, chemicals, sun, and autoimmune diseases); or

preventing cell death of bone marrow cells in myelodysplastic syndromes (MDS); or

preventing and/or treating pancreatitis; or

preventing and/or treating respiratory syndrome; or

preventing and/or treating osteoarthritis, rheumatoid arthritis, psoriasis, glomerulonephritis, atherosclerosis, and graft versus host disease; or

preventing and/or treating disease states associated with an increase of apoptosis.

Other features of the invention will become apparent in the course of the following descriptions of exemplary embodiments which are given for illustration of the invention and are not intended to be limiting thereof.

### EXAMPLES

### Example 1

### In Vitro Activity

The in vitro caspase-2 inhibition activity of 2-quinolinylcarbonyl-(S)-Valinyl-(S)-Aspartyl (methyl ester)-(S)-Valinyl-(S)-Alanyl-(R,S)-Aspartyl (methyl ester) 2,3,5,6-tetrafluorophenyl ester (TRP 701) in comparison to other compounds was evaluated according to the following protocol. Samples of human recombinant caspase-2 (25-50U, BIOMOL, Plymouth, Pa., USA) were pre-incubated for 30 minutes with inhibitors (0.001 to 2 μM) in final 100 μl final assay buffer (50 mM HEPES, pH 7.4, 100 mM NaCI, 0.1% CHAPS, 10 mM DTT, 1 mM EDTA, 10% glycerol) and then mixed with 200 μM of the fluorogenic caspase substrates (BIOMOL) Ac-VDVAD-AMC. An IC₅₀ value corresponding to the concentration which inhibis 50% of caspase activity was determined from the dose-response sigmoid curve using the steady-state fluorescence approach. The cleavage of AMC-based substrate by human recombinant caspase-2 was measured after 2 hours at 37° C. on a fluorescence microplate reader by monitoring emission at 510 nm upon excitation at 405 nm.

The compounds used for comparative purposes were:

TRP 601 (quinoline-2-carbonyl-(S)-Val-(S)-Asp(OMe)-(S)-Val-(S)-Ala-(R,S)-Asp(OMe)-CH₂OC₆H₃(2,6-F₂)) identified with the code MZ77027;

rac-TRP 601, i.e. racemic TRP 601 identified with the code BS 03140;

M3 which is the compound 2-quinolinylcarbonyl-S-Valine, an inactive compound structurally related to TRP601; and

TRP 600 (quinoline-2-carbonyl-(S)-Val-(S)-Asp(OMe)-(S)-Val-(S)-Ala-(R,S)-Ala((R,S)-CH₂OC₆H₃(2,6-F₂)), another inactive compound structurally related to TRP601.

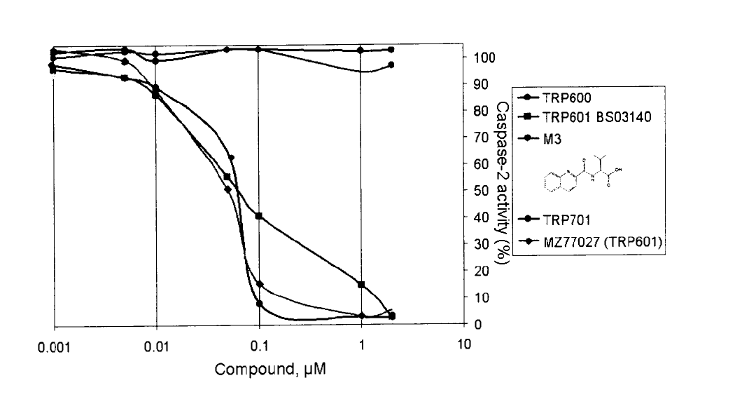

The dose response sigmoid curve is reported in FIG. 1. Curves are the mean of three independent runs.

TRP 701 turned out to be a selective caspase-2 inhibitor. It also turned out to be capable of inhibiting caspase-2 activity with an IC₅₀ similar to that of TRP 601, i.e. of around 60 to 90 nM.

### Example 2

### Impact on In Vivo Caspase-2 Kinetics and Brain Tissue Preservation Upon Pathological Condition

The activation kinetics of caspase-2 as well as efficacy based on histology parameters is evaluated though a convenient animal model, i.e., where caspase-2 is early active. Such a model allows a comparison of caspase-2 inhibitory potency and protective effect of 2-quinolinylcarbonyl-(S)-Valinyl-(S)-Aspartyl (methyl ester)-(S)-Valinyl-(S)-Alanyl-(R,S)-Aspartyl (methyl ester) 2,3,4,5,6-pentafluorophenyl ester (TRP 701) versus TRP601.

This model of neonatal stroke (cerebral ischemia with reperfusion) gives rise to an ipsilateral penumbra progression and leads to cortical injury (infarction) at 48 hours. This may in turn develop a cavity. This experimental model exhibits a real reperfusion step as noted in clinical syndrome and is relevant in term of brain maturation and blood-brain-barrier with the term human newborn. The pattern of lesion is very similar to that found in full term babies at birth or occurring in the following days/months after suffering at birth.

The experimental model is performed in 7 day-old rat pups (Wistar strain). Unilateral transient focal ischemia is induced in P7 Wistar rats of both sexes, as previously described (Renolleau Set al., Stroke, 1998 July; 29(7):1454-60, which is incorporated herein by reference in its entirety). Seven-day-old Wistar rats (Janvier, Le Genest-St-Isle, France) are anesthetized with chloral hydrate (i.p., 350 mg/kg) or gas anaesthesia. Briefly, the left Middle Cerebral Artery is coagulated at the inferior level of the cerebral vein, and then a clip is placed to occlude the left common carotid artery. After 50 minutes, the clip is removed and carotid blood flow restoration is verified by microscopy. During the surgical procedure, body temperature is maintained at 37 to 38° C. The test item is administered according to the route defined with the Sponsor at the time of reperfusion (corresponding to 1 hour post-ischemia onset). Importantly, vehicle and test item doses must be randomly assorted within a litter to take into account and to minimize differences of responses between litters. The pups are then transferred to an incubator (37° C.) until recovery, and then returned to their dams.

Rat pups are killed from reperfusion to 48 hours post-reperfusion, and their brains are removed. An infarct lesion (pale zone) appears progressively after reperfusion, reflecting the course of brain damages in the ipsilateral hemisphere, and should be reduced in case of efficient drug treatment.

To establish caspase-2 activity kinetics and determine whether level of caspase-2 activity after stroke is modified by TRP701 versus TRP701 treatment, enzymatic assay is performed from brains. Importantly, caspase-2 in vivo inhibition in the brain allows correlating with brain tissue protection. To proceed, caspase-2 activity must be measured in ischaemic penumbra only corresponding to the injured tissue. To conveniently measure any difference of activity, brains of rat pups (24 hours post-ischemia) are removed just after decapitation. Contralateral (CL) and ipsilateral (IL, containing the infarct) hemispheres are rapidly frozen and kept at −80° C. After thawing, the ipsilateral hemisphere is rapidly micro-dissected in order to take exclusively the penumbra area which exhibited more or less pronounced white colour according to the time of post-reperfusion. The corresponding area is also taken in the contralateral counterpart to measure the threshold of intrinsic caspase activity (caspase-2, C2; caspase-3, C3; Caspase-9, C9). Each tissue is then quickly processed in ice. The sample (1 to 2 mm³) is put into a glass tube containing 800 μl of buffer B (HEPES 10 mM pH 7.4, KCl 42 mM, MgCl2 5 mM, DTT 1 mM, CHAPS 0.5%, EDTA 0.1 mM) extemporaneously supplemented with protease inhibitors: PMSF 1 mM, leupeptin 1 μg/ml, pepstatin A 1 μg/ml, cytochalasin B 1 μM, chymopapain 10 μg/ml, antipain 1 μg/ml. The tube is placed in an ice-bath and a manual crushing is performed with a glass Potter. The crushed tissues are then kept for 24 hours at −80° C. at least, prior to thawing and elimination of debris (10 minutes, 4° C., 2000 g). 100 μg of supernatant is diluted in the buffer A (specific caspase activity buffer) and incubated for 2 to 3 hours at 37° C. in presence of specific commercially available caspase substrates (50 μM): for Caspase-2 (C2), caspase-3 (C3) and caspase-9 (C9). Caspase activities are monitored by spectrofluorimetry (λex=380 nm; λem=465 nm).

To establish a protective effect of TRP 701, rat pups are killed at 48 hours post-reperfusion and their brains are removed. The brains are then fixed for 2 days in 4% PFA. 50 μm coronal brain sections are cut on a cryostat and collected on gelatin-coated slides. Eighteen sections from anterior striatum to posterior hippocampus (Bregma +3 mm to −6.5 mm) may be selected, taken at equally spaced 0.5 mm intervals. Lesion areas are measured on cresyl violet-stained sections using an image analyzer, and distances between respective coronal sections are used to calculate the infarct volume and the % infarct volume in the ipsilateral hemisphere (based on the Cavalieri principle).

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A compound of formula (I): 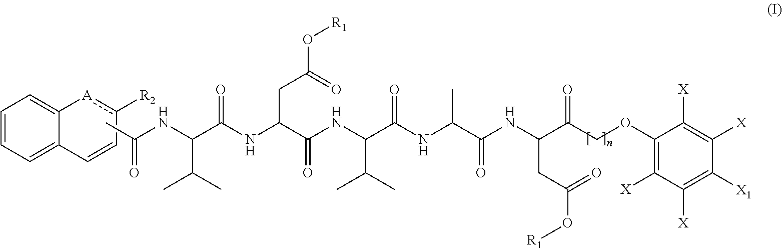
wherein:
n is 0 or 1;
A represents N, N(H), O, S, or N—O (i.e. N-oxide) [symbol]
adjacent to the group A can be a single bond when A is O or S or a single or double bond when A is N(H) or N, respectively;
each X may be the same or different from each other and is independently selected from halogen atoms;
X₁ represents H or a halogen atom;
each R₁ may be the same or different from each other and is independently selected from H and a linear or branched (C₁-C₄) alkyl group; and
R₂ is absent, when [symbol] is a double bond, and is H, when [symbol] is a single bond.
or a pharmaceutically acceptable salt thereof.

2. A compound or salt according to claim 1, wherein A is N and [symbol] is a double bond.

3. A compound or salt according to claim 1, which is a quinoline N-oxide.

4. A compound or salt according to claim 1, having formula (II): 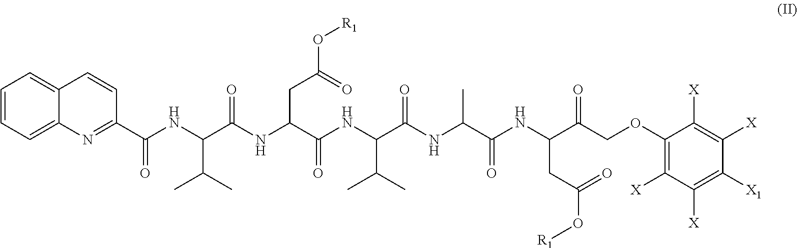
or a pharmaceutically acceptable salt or N-oxide of a compound of formula (II).

5. A compound or salt according to claim 1, wherein each X is a fluorine atom.

6. A compound or salt according to claim 5, wherein X₁ is H.

7. A compound or salt according to claim 1, wherein R_{I} is H.

8. A compound or salt according to claim 1, wherein R₁ is methyl or ethyl.

9. A compound or salt according to claim 1, which is 5-(2,3,5,6-tetrafluoro-phenoxy)-3(R,S)-{2(S)-[2(S)-(3-methoxycarbonyl-2(S)-{3-methyl-2(S)-[(quinoline-2-carbonyl)-amino]-butyrylamino}-propionylamino)-3-methyl-butyrylamino]-propionylamino}-4-oxo-pentanoic acid methyl ester or a pharmaceutically acceptable salt thereof.

10. A pharmaceutical composition, comprising a compound or pharmaceutically acceptable salt according to claim 1 and one or more pharmaceutically acceptable excipients.

11. A pharmaceutical composition, comprising a compound or pharmaceutically acceptable salt according to claim 2 and one or more pharmaceutically acceptable excipients.

12. A pharmaceutical composition, comprising a compound or pharmaceutically acceptable salt according to claim 3 and one or more pharmaceutically acceptable excipients.

13. A pharmaceutical composition, comprising a compound or pharmaceutically acceptable salt according to claim 4 and one or more pharmaceutically acceptable excipients.

14. A pharmaceutical composition, comprising a compound or pharmaceutically acceptable salt according to claim 5 and one or more pharmaceutically acceptable excipients.

15. A pharmaceutical composition, comprising a compound or pharmaceutically acceptable salt according to claim 6 and one or more pharmaceutically acceptable excipients.

16. A method for treating and/or preventing a disease in which caspase-2 activity is implicated, comprising administering an effective amount of a compound or salt according to claim 1 to a subject in need thereof.

17. A method according to claim 16, wherein said compound or salt is administered in an amount of 0.01 mg/kg of body weigh to 5 mg/kg of body weight.

18. A method according to claim 16, wherein said disease derives from a hypoxic-ischemic (H-I) brain damage.

19. A method according to claim 16, wherein said disease is a neonatal brain injury.

20. A method according to claim 16, wherein said disease is selected from the group consisting of perinatal arterial stroke, perinatal hypoxic-ischaemic encephalopathy, and periventricular leucomalacia in premature babies.
